(19) United States
(12) Patent Application Publication
(10) Pub. No.: US 2026/0263178 A1
(43) Pub. Date: Sep. 10, 2026

(54) SURGICAL ROBOTIC SYSTEM AND METHOD FOR CHANGING ALERT BEHAVIOR BASED ON SURGEON EXPERIENCE

(71) Applicant: Covidien LP

(72) Inventors: Jared N. Farlow, Los Angeles, CA (US); Donald E. Barry, Jr., Groton, MA (US); Scott Hopkinson, Concord, MA (US)

(21) Appl. No.: 19/165,753

(22) PCT Filed: Mar. 11, 2024
(86) PCT No.: PCT/IB2024/052346

§ 371 Date: Sep. 16, 2025

Related U.S. Application Data
(60) Provisional application No. 63/453,798, filed on Mar. 22, 2023.

(51) Int. Cl. A61B 34/37; A61B 34/00; G16H 40/63

(52) U.S. Cl. CPC A61B 34/37; A61B 34/74; A61B 34/77; G16H 40/63

(57) ABSTRACT

A surgeon console for a surgical robotic system includes a computer-readable media storing a plurality of alerts classified in a hierarchy of classes. The console includes a controller configured to receive an operator identifier associated with an operator of the surgeon console, receive a key performance indicator (KPI) value associated with the operator, enable adjustment of the plurality of alerts based on the KPI value, and monitor operation of the surgical robotic system based on the plurality of alerts.

### CROSS-REFERENCE TO RELATED APPLICATIONS

This application claims the benefit of U.S. Provisional Patent Application Ser. No. 63/453,798, filed Mar. 22, 2023, the entire content of which is incorporated herein by reference.

### BACKGROUND

Surgical robotic systems are currently being used in a variety of surgical procedures, including minimally invasive medical procedures. Some surgical robotic systems include a surgeon console controlling a surgical robotic arm and a surgical instrument having an end effector (e.g., forceps or grasping instrument) coupled to and actuated by the robotic arm. In operation, the robotic arm is moved to a position over a patient and then guides the surgical instrument into a small incision via a surgical port or a natural orifice of a patient to position the end effector at a work site within the patient's body. The surgical robotic systems are extremely complex and use a variety of safety features monitoring its operation. However, not all operators have the same level of experience with the robotic systems and certain safety may be distracting to certain operators.

### SUMMARY

According to one embodiment of the present disclosure, a surgeon console for a surgical robotic system is described. The surgeon console includes a computer-readable media storing a plurality of alerts classified in a hierarchy of classes. The console includes a controller configured to receive an operator identifier associated with an operator of the surgeon console, receive a key performance indicator (KPI) value associated with the operator, enable adjustment of the plurality of alerts based on the KPI value, and monitor operation of the surgical robotic system based on the plurality of alerts.

KPI (key performance indicators) may include warnings and alarms associated with the operation of the system. The concept being that a qualified and experienced end user of the system (such as the surgeon) can assign a warning level of a particular event to one of several severity levels. The system may limit how much the surgeon or end user can elevate or demote (reduce) the severity level of any given alarm, warning or KPI.

Implementations of the above embodiment may include one or more of the following features. According to one aspect of the above embodiment, the controller may be further configured to compare the KPI value to a KPI threshold. The controller may be also configured to enable the adjustment of the plurality of alerts in response to the KPI value being larger than the KPI threshold. The controller may be additionally configured to load default settings of the plurality of alerts in response to the KPI value being lower than the KPI threshold. The adjustment of the plurality of alerts may include changing a classification of at least one alert of the plurality of alerts. The controller may be also configured to automatically the change classification of at least one alert of the plurality of alerts based on the KPI value. The controller may be further configured to retrieve or calculate the KPI value from a database based on the operator identifier. The controller may be additionally configured to receive the operator identifier and the KPI value from an identification card.

According to another embodiment of the present disclosure, a surgical robotic system is described. The surgical robotic system includes a robotic arm having a surgical instrument. The system also includes a surgeon console having a handle controller configured to receive user input to move the robotic arm. The surgeon console also includes a computer-readable media storing a plurality of alerts classified in a hierarchy of classes. The surgeon console further includes a controller configured to receive an operator identifier associated with an operator of the surgeon console, receive a key performance indicator (KPI) value associated with the operator, enable adjustment of the plurality of alerts based on the KPI value, and monitor operation of the surgical robotic system based on the plurality of alerts.

Implementations of the above embodiment may include one or more of the following features. According to one aspect of the above embodiment, the controller may be further configured to adjust the user input by a scaling factor to control movement of the robotic arm. The controller may be also configured to adjust the scaling factor based on the KPI value. The controller may be additionally configured to compare the KPI value to a KPI threshold. The controller may be also configured to adjust the scaling factor in response to the KPI value being larger than the KPI threshold. The controller may be also configured to enable the adjustment of the plurality of alerts in response to the KPI value being larger than the KPI threshold. The controller may be further configured to load default settings of the plurality of alerts in response to the KPI value being lower than the KPI threshold.

According to a further embodiment of the present disclosure, a method for adjusting alert behavior based on operator experience is described. The method includes receiving at a surgeon console of a surgical robotic system an operator identifier associated with an operator of the surgeon console. The method further includes receiving at the surgeon console a key performance indicator (KPI) value associated with the operator and enabling adjustment of the plurality of alerts based on the KPI value. The method further includes monitoring operation of the surgical robotic system based on the plurality of alerts.

Implementations of the above embodiment may include one or more of the following features. According to one aspect of the above embodiment, the method may also include comparing the KPI value to a KPI threshold. The method may further include enabling the adjustment of the plurality of alerts in response to the KPI value being larger than the KPI threshold. The method may additionally include loading default settings of the plurality of alerts in response to the KPI value being lower than the KPI threshold. The adjustment of the plurality of alerts may include changing a classification of at least one alert of the plurality of alerts.

### BRIEF DESCRIPTION OF THE DRAWINGS

Various embodiments of the present disclosure are described herein with reference to the drawings wherein:

FIG. 1 is a schematic illustration of a surgical robotic system including a control tower, a console, and one or more surgical robotic arms each disposed on a mobile cart according to an embodiment of the present disclosure;

FIG. 2 is a perspective view of a surgical robotic arm of the surgical robotic system of FIG. 1 according to an embodiment of the present disclosure;

FIG. 3 is a perspective view of a mobile cart having a setup arm with the surgical robotic arm of the surgical robotic system of FIG. 1 according to an embodiment of the present disclosure;

FIG. 4 is a schematic diagram of a computer architecture of the surgical robotic system of FIG. 1 according to an embodiment of the present disclosure;

FIG. 5 is a plan schematic view of the surgical robotic system of FIG. 1 positioned about a surgical table according to an embodiment of the present disclosure;

FIG. 6 is a schematic diagram of a system for determining phases of a surgical procedure according to an embodiment of the present disclosure; and

FIG. 7 is a flow chart illustrating a method for adjusting operation of safety features based on surgeon experience.

### DETAILED DESCRIPTION

Embodiments of the presently disclosed surgical robotic system are described in detail with reference to the drawings, in which like reference numerals designate identical or corresponding elements in each of the several views.

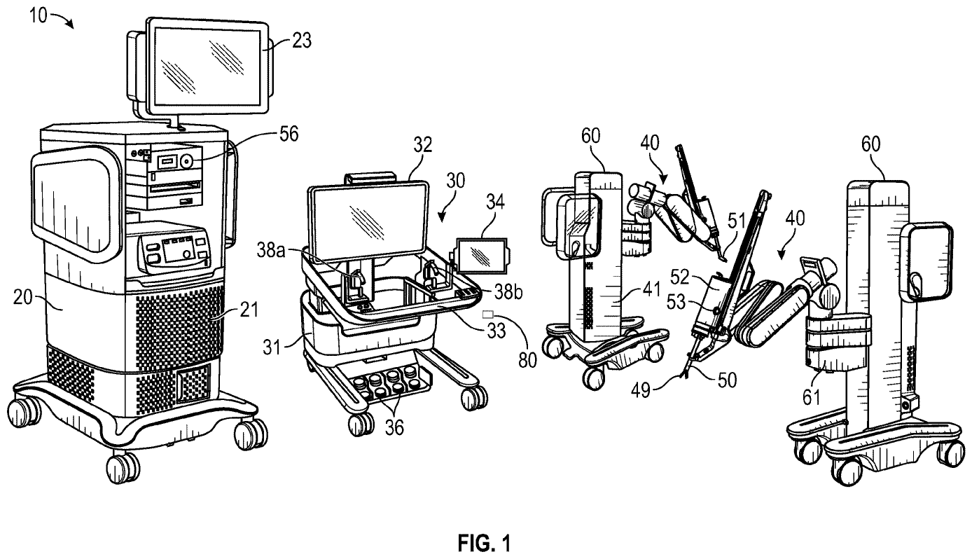

With reference to FIG. 1, a surgical robotic system 10 includes a control tower 20, which is connected to all of the components of the surgical robotic system 10 including a surgeon console 30 and one or more mobile carts 60. Each of the mobile carts 60 includes a robotic arm 40 having a surgical instrument 50 removably coupled thereto. The robotic arms 40 also couple to the mobile carts 60. The robotic system 10 may include any number of mobile carts 60 and/or robotic arms 40.

The surgical instrument 50 is configured for use during minimally invasive surgical procedures. In embodiments, the surgical instrument 50 may be configured for open surgical procedures. In further embodiments, the surgical instrument 50 may be an electrosurgical forceps configured to seal tissue by compressing tissue between jaw members and applying electrosurgical current thereto. In yet further embodiments, the surgical instrument 50 may be a surgical stapler including a pair of jaws configured to grasp and clamp tissue while deploying a plurality of tissue fasteners, e.g., staples, and cutting stapled tissue. In yet further embodiments, the surgical instrument 50 may be a surgical clip applier including a pair of jaws configured apply a surgical clip onto tissue.

One of the robotic arms 40 may include a laparoscopic camera 51 configured to capture video of the surgical site. The laparoscopic camera 51 may be a stereoscopic endoscope configured to capture two side-by-side (i.e., left and right) images of the surgical site to produce a video stream of the surgical scene. The laparoscopic camera 51 is coupled to an image processing device 56, which may be disposed within the control tower 20. The image processing device 56 may be any computing device configured to receive the video feed from the laparoscopic camera 51 and output the processed video stream.

The surgeon console 30 includes a first screen 32, which displays a video feed of the surgical site provided by camera 51 of the surgical instrument 50 disposed on the robotic arm 40, and a second screen 34, which displays a user interface for controlling the surgical robotic system 10. The first screen 32 and second screen 34 may be touchscreens allowing for displaying various graphical user inputs.

The surgeon console 30 also includes a plurality of user interface devices, such as foot pedals 36 and a pair of hand controllers 38a and 38b which are used by a user to remotely control robotic arms 40. The surgeon console further includes an armrest 33 used to support clinician's arms while operating the hand controllers 38a and 38b.

The control tower 20 includes a screen 23, which may be a touchscreen, and outputs on the graphical user interfaces (GUIs). The control tower 20 also acts as an interface between the surgeon console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgeon console 30, in such a way that robotic arms 40 and the surgical instrument 50 execute a desired movement sequence in response to input from the foot pedals 36 and the hand controllers 38a and 38b. The foot pedals 36 may be used to enable and lock the hand controllers 38a and 38b, repositioning camera movement and electrosurgical activation/deactivation. In particular, the foot pedals 36 may be used to perform a clutching action on the hand controllers 38a and 38b. Clutching is initiated by pressing one of the foot pedals 36, which disconnects (i.e., prevents movement inputs) the hand controllers 38a and/or 38b from the robotic arm 40 and corresponding instrument 50 or camera 51 attached thereto. This allows the user to reposition the hand controllers 38a and 38b without moving the robotic arm(s) 40 and the instrument 50 and/or camera 51. This is useful when reaching control boundaries of the surgical space.

Each of the control tower 20, the surgeon console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area network, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DC). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-1203 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

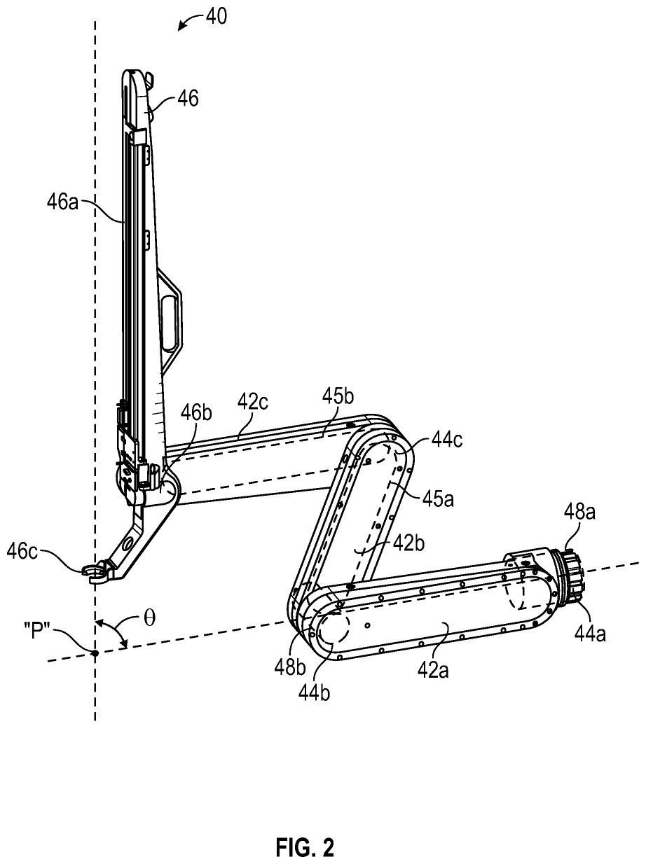

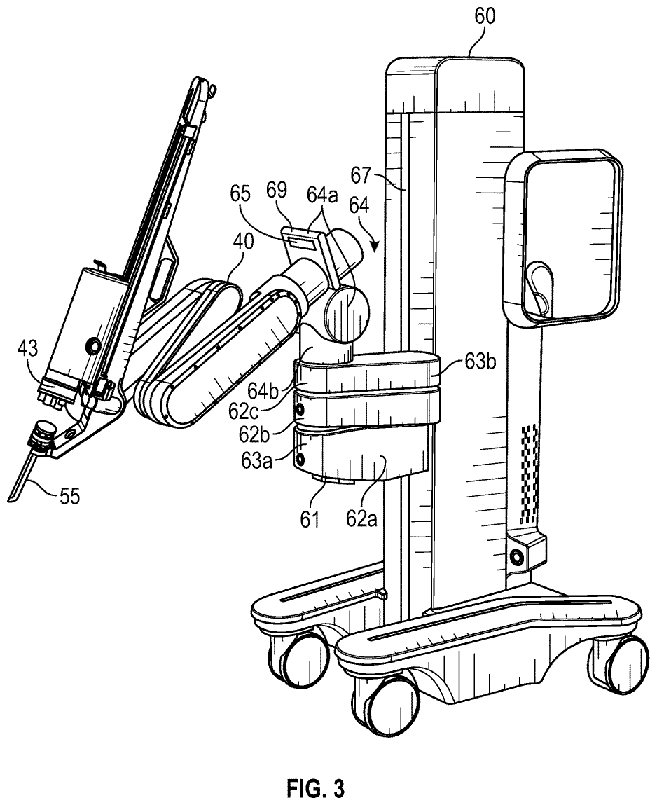

With reference to FIG. 2, each of the robotic arms 40 may include a plurality of links 42a, 42b, 42c, which are interconnected at joints 44a, 44b, 44c, respectively. Other configurations of links and joints may be utilized as known by those skilled in the art. The joint 44a is configured to secure the robotic arm 40 to the mobile cart 60 and defines a first longitudinal axis. With reference to FIG. 3, the mobile cart 60 includes a lift 67 and a setup arm 61, which provides a base for mounting of the robotic arm 40. The lift 67 allows for vertical movement of the setup arm 61. The mobile cart 60 also includes a screen 69 for displaying information pertaining to the robotic arm 40. In embodiments, the robotic arm 40 may include any type and/or number of joints.

The setup arm 61 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In embodiments, the robotic arm 40 may be coupled to the surgical table (not shown). The setup arm 61 includes controls 65 for adjusting movement of the links 62a, 62b, 62c as well as the lift 67. In embodiments, the setup arm 61 may include any type and/or number of joints.

The third link 62c may include a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

The actuator 48b of the joint 44b is coupled to the joint 44c via the belt 45a, and the joint 44c is in turn coupled to the joint 46b via the belt 45b. Joint 44c may include a transfer case coupling the belts 45a and 45b, such that the actuator 48b is configured to rotate each of the links 42b, 42c and a holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. In other words, the pivot point “P” is a remote center of motion (RCM) for the robotic arm 40. Thus, the actuator 48b controls the angle θ between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle θ. In embodiments, some or all of the joints 44a, 44b, 44c may include an actuator to obviate the need for mechanical linkages.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

With reference to FIG. 2, the holder 46 defines a second longitudinal axis and configured to receive an instrument drive unit (IDU) 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components an end effector 49 of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c. During endoscopic procedures, the instrument 50 may be inserted through an endoscopic access port 55 (FIG. 3) held by the holder 46. The holder 46 also includes a port latch 46c for securing the access port 55 to the holder 46 (FIG. 2).

The robotic arm 40 also includes a plurality of manual override buttons 53 (FIG. 1) disposed on the IDU 52 and the setup arm 61, which may be used in a manual mode. The user may press one or more of the buttons 53 to move the component associated with the button 53.

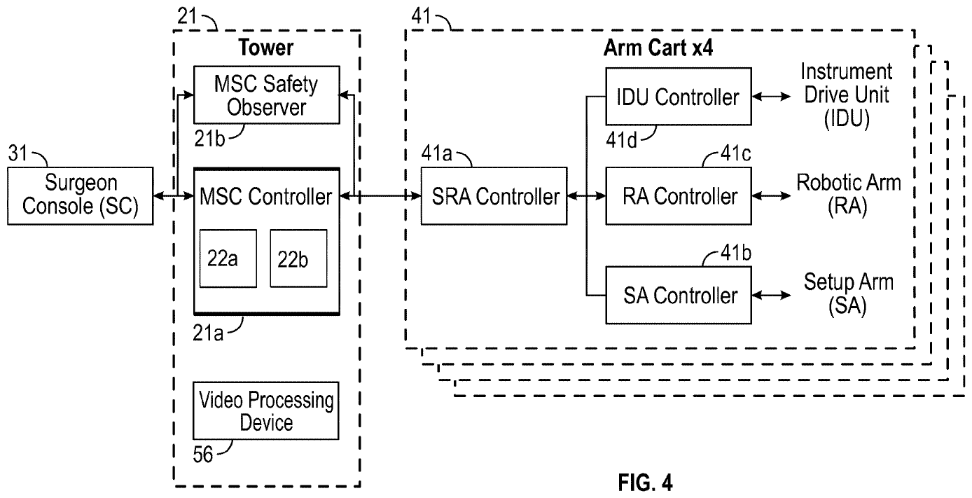

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgeon console 30 about the current position and/or orientation of the hand controllers 38a and 38b and the state of the foot pedals 36 and other buttons. The controller 21a processes these input positions to determine desired drive commands for each joint of the robotic arm 40 and/or the IDU 52 and communicates these to the computer 41 of the robotic arm 40. The controller 21a also receives the actual joint angles measured by encoders of the actuators 48a and 48b and uses this information to determine force feedback commands that are transmitted back to the computer 31 of the surgeon console 30 to provide haptic feedback through the hand controllers 38a and 38b. The safety observer 21b performs validity checks on the data going into and out of the controller 21a and notifies a system fault handler if errors in the data transmission are detected to place the computer 21 and/or the surgical robotic system 10 into a safe state.

The controller 21a is coupled to a storage 22a, which may be non-transitory computer-readable medium configured to store any suitable computer data, such as software instructions executable by the controller 21a. The controller 21a also includes transitory memory 22b for loading instructions and other computer readable data during execution of the instructions. In embodiments, other controllers of the system 10 include similar configurations.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the mobile cart 60, the robotic arm 40, and the IDU 52. The main cart controller 41a also communicates actual joint angles back to the controller 21a.

Each of joints 63a and 63b and the rotatable base 64 of the setup arm 61 are passive joints (i.e., no actuators are present therein) allowing for manual adjustment thereof by a user. The joints 63a and 63b and the rotatable base 64 include brakes that are disengaged by the user to configure the setup arm 61. The setup arm controller 41b monitors slippage of each of joints 63a and 63b and the rotatable base 64 of the setup arm 61, when brakes are engaged or can be freely moved by the operator when brakes are disengaged, but do not impact controls of other joints. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

The robotic arm 40 is controlled in response to a pose of the hand controller controlling the robotic arm 40, e.g., the hand controller 38a, which is transformed into a desired pose of the robotic arm 40 through a hand eye transform function executed by the controller 21a. The hand eye function, as well as other functions described herein, is/are embodied in software executable by the controller 21a or any other suitable controller described herein. The pose of one of the hand controllers 38a may be embodied as a coordinate position and roll-pitch-yaw (RPY) orientation relative to a coordinate reference frame, which is fixed to the surgeon console 30. The desired pose of the instrument 50 is relative to a fixed frame on the robotic arm 40. The pose of the hand controller 38a is then scaled by a scaling function executed by the controller 21a. In embodiments, the coordinate position may be scaled down and the orientation may be scaled up by the scaling function. In addition, the controller 21a may also execute a clutching function, which disengages the hand controller 38a from the robotic arm 40. In particular, the controller 21a stops transmitting movement commands from the hand controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the hand controller 38a and is then passed by an inverse kinematics function executed by the controller 21a. The inverse kinematics function calculates angles for the joints 44a, 44b, 44c of the robotic arm 40 that achieve the scaled and adjusted pose input by the hand controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

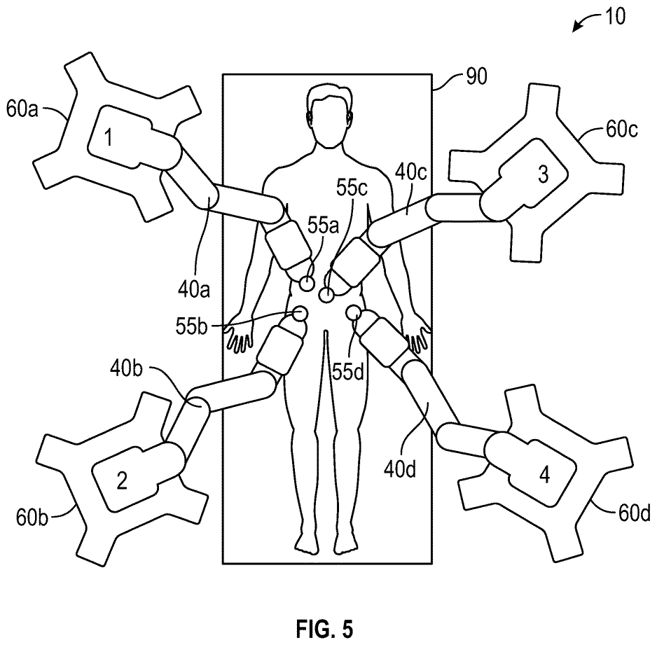

With reference to FIG. 5, the surgical robotic system 10 is setup around a surgical table 90. The system 10 includes mobile carts 60a-d, which may be numbered “1” through “4.” During setup, each of the carts 60a-d are positioned around the surgical table 90. Position and orientation of the carts 60a-d depends on a plurality of factors, such as placement of a plurality of access ports 55a-d, which in turn, depends on the surgery being performed. Once the port placement is determined, the access ports 55a-d are inserted into the patient, and carts 60a-d are positioned to insert instruments 50 and the laparoscopic camera 51 into corresponding ports 55a-d.

During use, each of the robotic arms 40a-d is attached to one of the access ports 55a-d that is inserted into the patient by attaching the latch 46c (FIG. 2) to the access port 55 (FIG. 3). The IDU 52 is attached to the holder 46, followed by the SIM 43 being attached to a distal portion of the IDU 52. Thereafter, the instrument 50 is attached to the SIM 43. The instrument 50 is then inserted through the access port 55 by moving the IDU 52 along the holder 46. The SIM 43 includes a plurality of drive shafts configured to transmit rotation of individual motors of the IDU 52 to the instrument 50 thereby actuating the instrument 50. In addition, the SIM 43 provides a sterile barrier between the instrument 50 and the other components of robotic arm 40, including the IDU 52. The SIM 43 is also configured to secure a sterile drape (not shown) to the IDU 52.

A surgical procedure may include multiple phases, and each phase may include one or more surgical actions. As used herein, the term “phase” represents a surgical event that is composed of a series of steps (e.g., closure). A “surgical action” may include an incision, a compression, a stapling, a clipping, a suturing, a cauterization, a sealing, or any other such actions performed to complete a phase in the surgical procedure. A “step” refers to the completion of a named surgical objective (e.g., hemostasis). During each step, certain surgical instruments 50 (e.g., forceps) are used to achieve a specific objective by performing one or more surgical actions.

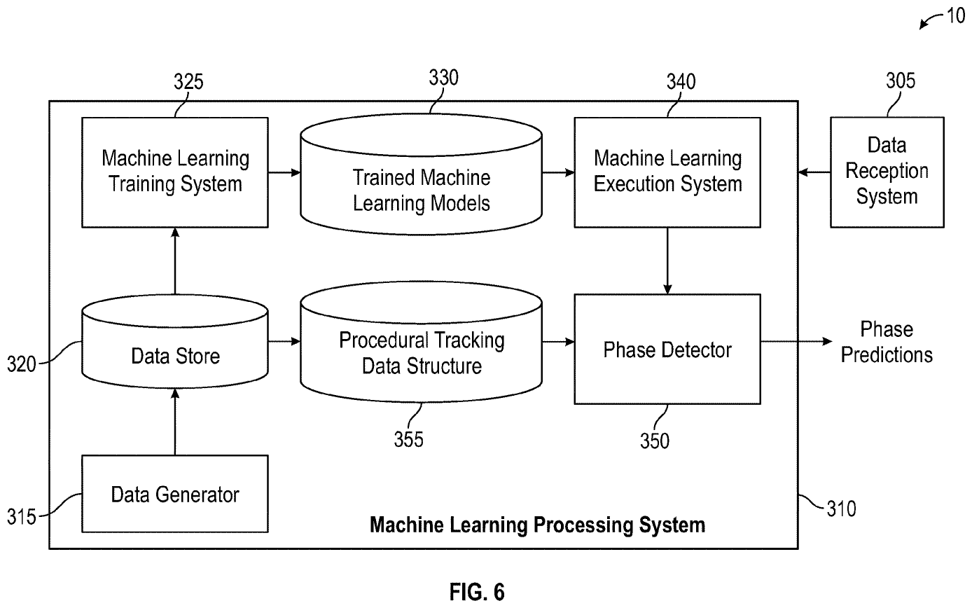

With reference to FIG. 6, the surgical robotic system 10 may include a machine learning (ML) processing system 310 that processes the surgical data using one or more ML models to identify one or more features, such as surgical phase, instrument, anatomical structure, etc., in the surgical data. The ML processing system 310 includes a ML training system 325, which may be a separate device (e.g., server) that stores its output as one or more trained ML models 330. The ML models 330 are accessible by a ML execution system 340. The ML execution system 340 may be separate from the ML training system 325, namely, devices that “train” the models are separate from devices that “infer,” i.e., perform real-time processing of surgical data using the trained ML models 330.

System 10 includes a data reception system 305 that collects surgical data, including the video data and surgical instrumentation data. The data reception system 305 can include one or more devices (e.g., one or more user devices and/or servers) located within and/or associated with a surgical operating room and/or control center. The data reception system 305 can receive surgical data in real-time, i.e., as the surgical procedure is being performed.

The ML processing system 310, in some examples, may further include a data generator 315 to generate simulated surgical data, such as a set of virtual images, or record the video data from the video processing device 56, to train the ML models 330 as well as other sources of data, e.g., user input, arm movement, etc. Data generator 315 can access (read/write) a data store 320 to record data, including multiple images and/or multiple videos.

The ML processing system 310 also includes a phase detector 350 that uses the ML models to identify a phase within the surgical procedure (“procedure”). Phase detector 350 uses a particular procedural tracking data structure 355 from a list of procedural tracking data structures. Phase detector 350 selects the procedural tracking data structure 355 based on the type of surgical procedure that is being performed. In one or more examples, the type of surgical procedure is predetermined or input by user. The procedural tracking data structure 355 identifies a set of potential phases that may correspond to a part of the specific type of procedure.

In some examples, the procedural tracking data structure 355 may be a graph that includes a set of nodes and a set of edges, with each node corresponding to a potential phase. The edges may provide directional connections between nodes that indicate (via the direction) an expected order during which the phases will be encountered throughout an iteration of the procedure. The procedural tracking data structure 355 may include one or more branching nodes that feed to multiple next nodes and/or may include one or more points of divergence and/or convergence between the nodes. In some instances, a phase indicates a procedural action (e.g., surgical action) that is being performed or has been performed and/or indicates a combination of actions that have been performed. In some instances, a phase relates to a biological state of a patient undergoing a surgical procedure. For example, the biological state may indicate a complication (e.g., blood clots, clogged arteries/veins, etc.), pre-condition (e.g., lesions, polyps, etc.). In some examples, the ML models 330 are trained to detect an “abnormal condition,” such as hemorrhaging, arrhythmias, blood vessel abnormality, etc.

The phase detector 350 outputs the phase prediction associated with a portion of the video data that is analyzed by the ML processing system 310. The phase prediction is associated with the portion of the video data by identifying a start time and an end time of the portion of the video that is analyzed by the ML execution system 340. The phase prediction that is output may include an identity of a surgical phase as detected by the phase detector 350 based on the output of the ML execution system 340. Further, the phase prediction, in one or more examples, may include identities of the structures (e.g., instrument, anatomy, etc.) that are identified by the ML execution system 340 in the portion of the video that is analyzed. The phase prediction may also include a confidence score of the prediction. Other examples may include various other types of information in the phase prediction that is output. The predicted phase may be used by the controller 21a to determine which alerts are activated based on the phase and operator experience, as detailed below.

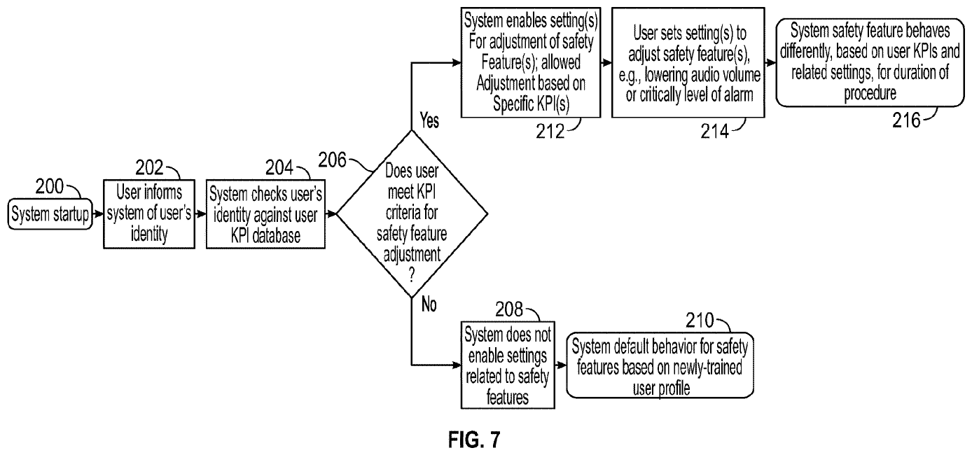

With reference to FIG. 7, a method for adjusting operation of safety features based on surgeon experience is shown. The method may be implemented as software instructions stored in a non-transitory storage media (e.g., local storage 22a, cloud storage, etc.) executable by one or more processors (e.g., controller 21a or any other suitable CPU, GPU, etc.). At step 200, the system 10 is started up, which includes powering on the control tower 20 and connecting and powering on the peripheral components. In particular, the surgeon console 30 and the robotic arms 40 are connected to the control tower 20 and are powered on. The robotic arms 40 may also be moved to designated positions as shown in FIG. 5.

At step 202, identity of the operator is provided to the system 10 (e.g., an operator logs into the surgeon console 30). This may be performed using any suitable method, such as an identification card 80 (FIG. 1) that is read by the surgeon console 30, inputting username and password, biometric authentication, etc. The ID card 80 may be an RFID card, an integrated circuit card (e.g., smart card), USB dongle, etc. and may include any storage medium configured to communicate with the surgeon console 30 in a wireless or wired manner. The system 10 has access to an operator database, which may be stored locally in a memory of the system 10 or remotely, e.g., cloud storage. The operator database includes a list of authorized operators and operator parameters and preferences, such as configuration parameters for the surgeon console 30, e.g., ergonomic parameters, GUI settings, etc. In embodiments, KPI value may be provided on a different platform from the robotic system. For example, the database(s) with KPIs and associating them with the operator could be on servers communicating with a smartphone app, and the smartphone could simply communicate the operator's personal KPI values (or even a resulting but reframed factor like a letter grade) to the system via Bluetooth or another communication method.

The operator database may also store key performance indicator (KPI) values of authorized operators. In embodiments, the KPI value may be stored on the ID card 80 or another storage device used during login. The KPI value may be a numerical value that is calculated, e.g., by the system 10 or by a server etc., based on the experience and/or proficiency level of the operator, and may be based on total operating time, efficiency factors, error rates, training, and other parameters. Training parameters may include the number of training modules completed as well as the performance of those training modules. Thus, the KPI value may increase as the user becomes more experienced and efficient. The KPI value may decrease due to an increase in error rate or other adverse performance factors. Parameters used in calculating the KPI value may also be weighted using relative complexity of the procedure and the patient. Thus, an efficient and error-free performance in a complicated procedure and/or on patient results in a higher increase of the KPI value when compared to the same performance in a simpler procedure and/or healthy patient. The KPI value and thresholds may be reversed, where a lower value indicates proficiency and vice versa, with the lower thresholds being indicative of proficiency.

The KPI value may be calculated using an artificial intelligence or machine learning (AI/ML) algorithm. The AI/ML algorithm may be based on statistical ML that is configured to a develop a statistical model and draw inferences therefrom. As more training data is provided, the system adjusts the statistical model and improves its ability to analyze or make predictions. Suitable statistical ML models include, but are not limited to, linear regression, logistic regression, decision trees, random forest, Naïve Bayes, ensemble methods, support vector machines, K-Nearest Neighbor, and the like. In further embodiments, the AL/ML algorithm may be a deep learning algorithm that incorporates neural networks in successive layers. Suitable deep learning models include, but are not limited to, convolutional neural network, recurrent neural network, deep reinforcement network, deep belief network, transformer network, and the like. The input provided to train the models may be previously collected operation data of the system 10, including operator inputs (e.g., number and type of operator inputs through the surgeon console 30), procedure duration, number and type of alerts and errors, etc.

At step 204, the system 10 retrieves or calculates the KPI value and compares the operator's KPI value to a threshold KPI value used in adjusting various safety-related (or risk-related) features e.g. alerts, settings affecting robotic arm movement etc. The system 10 is configured to continuously monitor operation, including user inputs, system events, etc. and determining whether the any of the events and inputs present a risk. The system 10 also classifies alerts in a hierarchy of different classes based on the degree of risk to the system 10, staff, and/or the patient, e.g., low, medium, high, etc. Low alerts denote that caution should be exercised in response to a detected event, medium alerts denote a warning and require intervention before continuing the procedure, and high alerts denote a danger and may require stopping of the procedure. Each of the different classes may include one or more corresponding responses from the operator and/or staff.

Exemplary low level alerts may include, but are not limited to:
Arm calibration failure. Retry calibration (RETRY), or touch IGNORE & CONTINUE to continue without this arm. Touch DISMISS to acknowledge.
Arm calibration failed. Disconnect and reconnect arm to retry calibration, or replace arm and contact support.
Two endoscopes not allowed. Detach one to enable surgeon console control.
Instrument was attached with tip below port. Insertion disabled. Withdraw instrument and check for instrument damage.
Arm communication failure; arm display not updating.
Alarm system failure. Withdraw instruments and discontinue system use.
Surgeon console display communication failure; surgeon console displays not updating. Check OR team interactive display for instructions.
Insertion disabled. Dock arm to enable insertion.
Arm fulcrum point has slipped. Reposition arm to alleviate port tension and clear alarm.
Instrument not ready to withdraw. Double click instrument drive unit button to enable withdraw.
Instrument withdrawal failed. Remove instrument and port together, inspect for damage. Touch DISMISS to acknowledge.
Arm over-temperature. Check arm and cart vents and remove obstructions.
Arm startup error. Reconnect failed arm to retry, or remove arm from use. Touch DISMISS to acknowledge.
Arm laser alignment button failure. Once un-braked, arm must be removed from use. Please contact support.
Arm startup error. Unplug and reconnect arm. If error reoccurs, remove arm from use; contact support.
Arm must be un-docked, un-braked and re-braked. If error persists, replace arm and contact support.
Arm calibration error. Make sure no port docked or buttons pressed. Retry, reconnect arm, or replace arm.
Sterile interface module not connected or non-functional. Arm sterile barrier may be open.
Instrument error. Detach instrument; clean and dry attachment contact surfaces. If error persists, discard instrument.
Error reading instrument. Remove and reattach instrument. If error persists, remove arm from use and contact support.
Two energy instruments of same type not allowed. Insertion disabled.
Surgeon console error. If error persists longer than 30 seconds, restart console using red AC power switch.
Surgeon console high temperature. Check console vents and remove obstructions.
Surgeon console foot pedal LED failure. Pedal lights may be off or color may be incorrect.
Surgeon console calibration error. Restart surgeon console using AC power switch. Touch DISMISS to acknowledge.
Surgeon console communication lost. Check console data cable. If error persists, restart surgeon console.
Ergonomic adjustment failure. Ergonomic adjustment disabled. Please contact support.
Hand controller error. Center and straighten in workspace, and remove hands. If error persists, discontinue surgeon console use.
Partial alarm system failure. Alarm audio, arm/cart LEDs, and/or arm displays may have failed. Touch DISMISS to resume use.
Alarm system test. If you heard an alarm tone, press YES. If you did NOT, please discontinue system use and contact support.
Tower recoverable error. Touch DISMISS to resume use.
Tower over-temperature.
OR team display failure. Discontinue system use. If error reoccurs, contact support. Touch DISMISS to acknowledge.
Endoscope error. Reconnect endoscope data cable. If error persists, restart endoscope system. Please contact support.
Unsupported endoscope. Endoscope functions may not work correctly. See user manual for supported endoscopes.
Endoscope disconnected from endoscope system. Reconnect, or withdraw and replace endoscope.
System startup failure. Touch SHUT DOWN or press blue power button to shut down and restart.
Endoscope configuration error. Restart system to retry, or discontinue system use and contact support.

Exemplary medium level alerts may include, but are not limited to:
Cart column failure. Remove arm from use; use column mechanical release if needed. Contact support.
Non-recoverable arm error. Follow other arm-specific notifications, or remove arm from use and unplug arm to continue.
Arm error. If instrument inserted, use mechanical releases to withdraw. If no instrument, unplug and re-plug arm.
Arm error. Withdraw instrument, unplug and reconnect arm. If error reoccurs, remove arm from use; contact support.
External force or collision detected. Resolve external force or collision to regain surgeon control.
Cart base LED light failure. Watch instrument drive unit LEDs.
Surgeon control of arm temporarily disabled. If condition persists, remove arm from use.
Arm communication error. Regain surgeon control. If error reoccurs, discontinue use of arm and contact support.
Arm failure. Use mechanical releases to withdraw. Unplug arm; contact support. Press DISMISS to acknowledge.
Arm system pause button failure. Use other system pause buttons if needed. If error reoccurs, contact support.
Surgeon console error. Restart console using red AC power switch. If error reoccurs, restart system or discontinue system use.
Surgeon console failure. Withdraw instruments and discontinue system use. Contact support. Touch DISMISS to acknowledge.
Surgeon console communication lost. Check data cable or restart console using red AC power switch. Touch DISMISS to acknowledge.
Surgeon console image error. If condition persists, restart surgeon console using red AC power switch.
Surgeon console non-recoverable error. Restart surgeon console. If error reoccurs, continue from bedside or discontinue system use.
Surgeon glasses error. Look at screen with only 1 pair of surgeon glasses. If error persists, discontinue surgeon console use.
Monopolar energy enabled; not attached to arm.
Bipolar energy enabled; not attached to arm.
Monopolar energy enabled outside patient.
Bipolar energy enabled outside patient.
Monopolar energy enabled on reserve arm.
Bipolar energy enabled on reserve arm.
Multiple monopolar instruments attached and enabled. Detach a Monopolar instrument and/or deactivate Monopolar energy.
Multiple bipolar instruments attached and enabled. Detach a bipolar instrument and/or deactivate bipolar energy.
Backup battery low; surgeon control disabled. Continue from bedside or discontinue system use. Touch DISMISS to acknowledge.
Backup battery charge low. Withdraw instruments and discontinue system use until fully charged.
Surgeon console system pause button failure. Use other pause buttons for remainder of procedure. Touch DISMISS to acknowledge.
Tower power system failure. Withdraw instruments, discontinue use of system and power system off. Please contact support.
System non-recoverable error; surgeon control disabled. Use bedside control to withdraw instruments and discontinue system use.
System paused. Touch DISMISS to resume use.
OR team display error. If error persists, withdraw instruments, discontinue system use, and contact support.
Electrosurgical generator non-recoverable error. Energy not available for rest of procedure. Touch DISMISS to acknowledge.
Tower arm cart cable socket failure. Use mechanical releases to withdraw, remove arm from use. Discontinue use of tower socket.
Electrosurgical generator non-recoverable error. Replace generator. Touch DISMISS to acknowledge.
Endoscope failure. Restart endoscope system, or withdraw instruments and discontinue system use. Please contact support.
Tower high temperature. Check tower vents and remove obstructions, or withdraw instruments and discontinue system use.
Tower system pause button failure. Use other system pause buttons for remainder of procedure. Touch DISMISS to acknowledge.
Arm error.
Sterile interface module expired. Detach and replace, re-drape if necessary.

Exemplary high level alerts, may include, but are not limited to:
Arm failure. Check for unintended arm motion. Use mechanical releases to withdraw; replace arm. Contact support.
Arm cart unbraked but docked. Set cart brake.
Cart column mechanical release active. Watch fulcrum point. Touch DISMISS to resume use.
Sterile interface module disconnected; arm sterile barrier open. Touch DISMISS to acknowledge.
Instrument drive unit mechanical release active. Withdraw instrument; remove arm from use. Contact support.
Arm undocked. Check port latch. Touch DISMISS to resume use.
Instrument error. Withdraw, detach and reattach instrument. If error reoccurs, replace instrument.
Instrument error. Withdraw normally. If withdrawal fails, remove instrument/port together. Replace instrument to resume.
Incorrect arm movement. Check arm for collision or object pushing on arm. Touch DISMISS to resume use.
Arm contact sensor activated, arm paused. Touch DISMISS to resume use.
Arm error. If instrument inserted, use mechanical releases to withdraw. If no instrument, unplug and re-plug arm.
Monopolar energy activated outside patient. Touch DISMISS to acknowledge.
Bipolar energy activated outside patient. Touch DISMISS to acknowledge.
Monopolar energy activated; not attached to arm. Touch DISMISS to acknowledge.
Bipolar energy activated; not attached to arm. Touch DISMISS to acknowledge.
Monopolar energy activated on uncontrolled arm. Touch DISMISS to acknowledge.
Bipolar energy activated on uncontrolled arm. Touch DISMISS to acknowledge.
Monopolar energy activated during bedside control. Touch DISMISS to acknowledge.
Bipolar energy activated during bedside control. Touch DISMISS to acknowledge.
Monopolar energy activated with multiple Monopolar instruments attached. Touch DISMISS to acknowledge.
Bipolar energy activated with multiple Bipolar instruments attached. Touch DISMISS to acknowledge.
Backup battery charge low; surgeon control disabled. Use bedside control to withdraw instruments and discontinue system use.
Total power loss approaching. Withdraw all instruments as soon as possible.
System failure. Check for unintended arm motion. Withdraw instruments and discontinue system use. Please contact support.
System AC power interrupted. Battery backup active. Check power connections, withdraw instruments and discontinue system use.
AC power interrupted. Battery backup active. Check power cords or withdraw instruments and discontinue system use.
Endoscope image frozen. Check endoscope system. If condition persists, withdraw instruments and discontinue system use.

At step 208, the system 10 checks if the operator's KPI value is a below a KPI threshold, i.e., due to lack of experience with the system 10. As described above, the KPI threshold denotes a rating of an experienced and/or proficient operator and may vary based on the type of the procedure being performed or other parameters and is loaded by the system 10 during initialization. If so, at step 210, the system 10 does not enable modification of alert settings and at step 212 the system 10 loads the unaltered alerts configuration, i.e., default alert settings, such that alerts are output in response to detected events.

If the operator's KPI value is above the KPI threshold, then the system 10 enables adjustment of safety features at step 214. The adjustment may be performed through a GUI displayed on one of the screens 23, 32, 34. The GUI may display alerts organized in different groups and/or allowing for toggling of individual alerts. In embodiments, the operator's preferred alert settings may be saved in the ID card 80 or otherwise loaded by the system 10. In further embodiments, the alert settings may be adjusted automatically based on the operator's KPI value.

At step 216, the adjusted settings are loaded by the controller 21a for the duration of the procedure. The controller 21a monitors events during various phases of the procedure and outputs alerts based on the loaded alerts, i.e., modified or unmodified. Certain safety settings would not be modified as they provide key safeguards for operation of the system 10.

In embodiments, if the usage data for newly trained operators, i.e., those with low KPI values, commit a particular use error at a certain frequency and operators with higher KPI values make the same use error at a lower rate, then a various input control parameters may be adjusted. Adjusted parameters include instrument motion scaling factor and elimination of additional enabling steps prior to critical inputs, e.g., electrosurgical activation. Thus, for operators having a KPI value above the threshold, the system 10 may allow the operator to set the scaling factor higher than the default scaling factor.

Described below are additional modifications of the safety alerts that may be made. Regarding high level alerts, such as mechanical release of the IDU 52 is active, the robotic arm 40 is moved incorrectly, activation of electrosurgical instruments outside the patient or while not attached to the robotic arm 40, the system 10 is reconfigured to allow bedside team to lower the alert to medium level.

Certain high level alerts may be adjusted to a medium level only after specific conditions are met. Thus, high level alerts for activation of electrosurgical instruments on uncontrolled arm, may be switched to a medium level once the operator has controlled the system 10 for a preset period of time without activating electrosurgical instruments on a reserved arm.

Another high level alert that may be adjusted is related to interruption in AC power to the system 10, which activates backup power systems and requires the system 10 to be undocked and instruments extracted, etc. within a preset time period, e.g., 5 minutes. This alert may be switched to a medium level based on average time the operating staff took to perform this action in response to previous power interruptions, e.g., the average time for undocking was 2 minutes. In further embodiments, the preset time period may be adjusted by the operator.

Certain instruments 50, such as electrosurgical instruments, are operated in a press-to-enable manner to prevent accidental activation. Certain medium level alerts, such as enabling delivery of electrosurgical energy while the instrument 50 is not attached to the robotic arm 40, outside the patient, or on a reserve robotic arm 40, etc. may allow the operator to turn off such press-to-enable features based on the recorded number of mistaken pedal taps during a preset period of time. This parameter may be included in the KPI value. The operator or the controller 21a may also adjust other parameters of the alerts, such as sound level and type of the sound and in visual alerts that are displayed on the GUI the color, size, and other parameters of the alerts.

It will be understood that various modifications may be made to the embodiments disclosed herein. Therefore, the above description should not be construed as limiting, but merely as exemplifications of various embodiments. Those skilled in the art will envision other modifications within the scope and spirit of the claims appended thereto.

What is claimed is:

1. A surgeon console for a surgical robotic system comprising:
a computer-readable media storing a plurality of alerts classified in a hierarchy of classes; and
a controller configured to:
receive an operator identifier associated with an operator of the surgeon console;
receive a key performance indicator (KPI) value associated with the operator;
enable adjustment of the plurality of alerts based on the KPI value; and
monitor operation of the surgical robotic system based on the plurality of alerts.

2. The surgeon console according to claim 1, wherein the controller is further configured to compare the KPI value to a KPI threshold.

3. The surgeon console according to claim 2, wherein the controller is further configured to: enable the adjustment of the plurality of alerts in response to the KPI value being larger than the KPI threshold.

4. The surgeon console according to claim 2, wherein the controller is further configured to load default settings of the plurality of alerts in response to the KPI value being lower than the KPI threshold.

5. The surgeon console according to claim 1, wherein the adjustment of the plurality of alerts includes changing a classification of at least one alert of the plurality of alerts.

6. The surgeon console according to claim 1, wherein the controller is further configured to automatically change the classification of at least one alert of the plurality of alerts based on the KPI value.

7. The surgeon console according to claim 1, wherein the controller is further configured to retrieve or calculate the KPI value from a database based on the operator identifier.

8. The surgeon console according to claim 1, wherein the controller is configured to receive the operator identifier and the KPI value from an identification card.

9. A surgical robotic system comprising:
a robotic arm including a surgical instrument;
a surgeon console including:
a handle controller configured to receive user input to move the robotic arm;
a computer-readable media storing a plurality of alerts classified in a hierarchy of classes; and
a controller configured to:
receive an operator identifier associated with an operator of the surgeon console;
receive a key performance indicator (KPI) value associated with the operator;
enable adjustment of the plurality of alerts based on the KPI value; and
monitor operation of the surgical robotic system based on the plurality of alerts.

10. The surgical robotic system according to claim 9, wherein the controller is further configured to adjust the user input by a scaling factor to control movement of the robotic arm.

11. The surgical robotic system according to claim 10, wherein the controller is further configured to adjust the scaling factor based on the KPI value.

12. The surgical robotic system according to claim 10, wherein the controller is further configured to compare the KPI value to a KPI threshold.

13. The surgical robotic system according to claim 12, wherein the controller is further configured to adjust the scaling factor in response to the KPI value being larger than the KPI threshold.

14. The surgical robotic system according to claim 12, wherein the controller is further configured to enable the adjustment of the plurality of alerts in response to the KPI value being larger than the KPI threshold.

15. The surgical robotic system according to claim 12, wherein the controller is further configured to load default settings of the plurality of alerts in response to the KPI value being lower than the KPI threshold.

16. A method for adjusting alert behavior based on operator experience, the method comprising:
receiving at a surgeon console of a surgical robotic system an operator identifier associated with an operator of the surgeon console;
receiving at the surgeon console a key performance indicator (KPI) value associated with the operator;
enabling adjustment of the plurality of alerts based on the KPI value; and
monitoring operation of the surgical robotic system based on the plurality of alerts.

17. The method according to claim 16, further comprising comparing the KPI value to a KPI threshold.

18. The method according to claim 17, further comprising enabling the adjustment of the plurality of alerts in response to the KPI value being larger than the KPI threshold.

19. The method according to claim 17, further comprising loading default settings of the plurality of alerts in response to the KPI value being lower than the KPI threshold.

20. The method according to claim 16, wherein the adjustment of the plurality of alerts includes changing a classification of at least one alert of the plurality of alerts.
